(19) United States
(12) Patent Application Publication
(10) Pub. No.: US 2006/0111600 A1
(43) Pub. Date: May 25, 2006

(54) Methods and apparatus for processing fuels

(76) Inventor: Lorrie Elmer Mecham, Mountain Home, ID (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/281,965

(22) Filed: Nov. 16, 2005

Related U.S. Application Data
(60) Provisional application No. 60/629,849, filed on Nov. 19, 2004.

(51) Int. Cl. C07C 7/00

(52) U.S. Cl. 585/800

(57) ABSTRACT

A fuel or other process fluid is cleansed by a) combining the fluid with water and an oxidizer; b) mixing the combination in a mixer having a volume V; c) separating the mixed combination into hydrophobic and hydrophilic phases; d) filtering the hydrophobic phase through a filter; e) removing water from the filtered hydrophobic phase to produce the cleaned fluid; and accomplishing steps a-d in a continuous manner that produces an output of the cleaned fuel at an average rate of at least 10V/hour. A centrifuge can optionally reduce water content of the cleaned fluid to no more than 5 ppm, more preferably no more than 1 ppm, and most preferably no more than 0.5 ppm.

### FIELD OF THE INVENTION

The field of the invention is processing of combustible fuels.

### BACKGROUND

Many fuels, especially reclaimed or renewable fuels such as biodiesel and fermentation derived ethanol, are heavily contaminated. Upon combustion, the contaminants contribute to air pollution, and eventually water and soil pollution. In some instances the contribution is strictly chemical, and in other instances the contribution can be some combination of chemical and physical interactions. For example, the current inventor has theorized that contaminant particles create hot spots that accelerate production of unburned and/or partially burned hydrocarbons, NOx, and particulates.

It is known to remove particulates from dirty fuels using a paper, resin, or other physical filter. Filters with micron sized holes, for example, can and have been used to reduce contaminants in fuels. But such filters are notoriously slow, and therefore to a large extent impractical.

It is also known to remove dissolved hydrophilic contaminants by washing a dirty fluid with water. As used herein, a dirty fluid is merely a fluid that is cleaned to produce a cleaned fluid. A biodiesel recipe employing a water wash, for example, can be found at http://localaction.biz/. In some instances contaminants can be rendered hydrophobic through chemical reactions. Sulfur contaminants, for example, can be removed by hydrogenating sulfur compounds to H₂S, and then removing the H₂S with amine solvents.

U.S. Pat. No. 4,314,902 to Bouk et al. (Feburary 1982), (the “'902 patent”), discloses a sophisticated solution to cleaning commercially available jet fuels that had been contaminated with microorganisms. This and all other cited materials are incorporated herein by reference in their entirety. Where a definition or use of a term in a reference, which is incorporated by reference herein, is inconsistent or contrary to the definition of that term provided herein, the definition of that term provided herein applies and the definition of that term in the reference does not apply.

The '902 patent teaches batch processing of the fuel by washing the contaminated fuel with an oxidizing agent (H₂O₂, cupric or ferric chloride), filtering the resultant by dumping in absorbent clay, and then decanting the cleaned fuel off the top. Unfortunately, that process is not commercially viable. The process is too slow, is not amenable to continuous process, and is wasteful in that a considerable amount of fuel remains with the clay and water sludge at the bottom of the processing vessel. Still further, there is no teaching or suggestion that the process can be applied to “raw” fuels, including for example, unfiltered biodiesel or fermentation derived ethanol, which can be heavily contaminated.

Thus, there is still a need for apparatus, systems, methods, and compositions for cleaning dirty fuels, and for marketing the cleaned fuels.

### SUMMARY OF THE INVENTION

The present invention provides apparatus, systems, methods, and compositions for marketing a fuel, comprising: providing a dirty fuel; using a process to clean the dirty fuel to produce a cleaned fuel; receiving written information from a competitor that the process is thought to be sufficient to reduce microbial contamination in biodiesel such that the cleaned biodiesel has no more than 100 CFUs/ml; and qualifying the cleaned fuel under an exhaust emissions standard. A CFU is a colony forming unit, as defined by the U.S. Department of Agriculture.

All types of fuels are contemplated, including especially fuels that contain one or more of diesel, gasoline, and ethanol. Of particular interest are fuels that include a blend of biodiesels, a biodiesel and a petroleum diesel, a biodiesel and a heating oil, and a gasoline with ethanol. Preferred fuels contain no more than 1000 CFUs/ml of microbial contamination, more preferably no more than 100 CFUs/ml of microbial contamination, and most preferably no more than 10 CFUs/ml of microbial contamination.

All recognized exhaust emissions standards are contemplated, including state, federal, and other standards, including for example standards announced or otherwise promulgated by SWRI (Southwest Research Institute) in San Antonio, Tex.

All practical aqueous solutions are contemplated to be used in washing the dirty fuel, including water in combination with hydrogen peroxide, ozone, and chromic acid, or any other suitable chemical oxidizer. Alternatively, the process can comprise partially oxidizing the dirty fuel using electrical energy, as for example by electrolyzing the fuel with immersed electrodes. Catalysts are also contemplated, including especially catalysts that increase or decrease the rate at which the fuel is oxidized, or that preferentially oxidize a contaminant in the fuel relative to the fuel itself. Preferred catalysts include copper, iron, magnesium, manganese, nickel, zinc, chromium or other metal ions.

Process controls are also contemplated. For example, it is contemplated that systems and methods can be implemented that sense an undesirable amount of a triol and a glyceride or other byproduct in a downstream portion of the cleaned fuel. Especially preferred process controls are at least partially automated, and thereby automatically modify a parameter of the process to reduce the byproduct in an upstream portion of the cleaned fuel. For example, if excess triol is detected, the system can automatically or otherwise increase hydrogen peroxide or mixing time.

Preferred methods of cleaning a fuel or other process fluid comprise: a) combining the process fluid with water and an oxidizer; b) mixing the combination in a mixer having a volume V; c) separating the mixed combination into hydrophobic and hydrophilic phases; d) filtering the hydrophobic phase through a filter; e) removing water from the filtered hydrophobic phase to produce the cleaned process fluid; and accomplishing steps a-d in a manner that produces an output of the cleaned fuel at an average rate of at least 10V/hour. Such processes can advantageously be accomplished in a substantially continuous manner, for example, with one or more of steps a-d being accomplished in a substantially continuous manner. In especially preferred methods, production lines can be arranged and operated that produce cleaned fuel at an average rate of at least 25V/hour, at least 35V/hour, and even at least 50V/hour.

The step of filtering preferably comprises passing the hydrophobic phase through a stationary consumable filter material. Preferred filter materials include one or more of paper, resin, activated carbon, and nano-graphene such as HRCM™ (available from SupraCarbonic, Inc, Irvine Calif.). Additional separation, especially to remove water, can be accomplished by centrifuging. Preferred centrifuges preferably reduce water content of the cleaned fuel to no more than 5 ppm, more preferably no more than 1 ppm, and most preferably no more than 0.5 ppm.

The apparatus, systems, methods, and compositions described herein can also be applied to cleaning oily materials that are not intended to be used as fuels. Examples include transformer oils and lubricants.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a schematic of a fuel or other material processing system according to aspects of the inventive subject matter.

### DETAILED DESCRIPTION

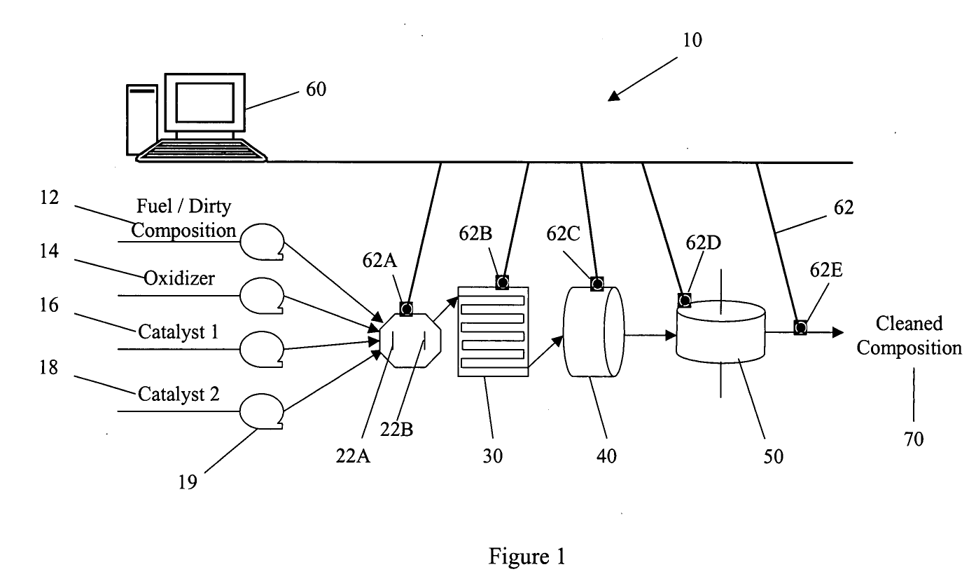

In FIG. 1, a purifying system 10 generally includes feed lines for input composition 12, oxidizer 14, first catalyst 16, second catalyst 18, a mixer 20, a separator 30, a filter 40, a centrifuge 50, computer controller 60, and an exit line 70. Pumps 19 provide motive force for movement of the corresponding compositions. In general, the vessels and various other conduction lines described herein are preferably made of stainless steel, glass, or other material that is non-reactive to the various chemicals.

Input composition 12 would usually be a liquid fuel, i.e. liquid that can reasonably be combusted to produce motive and/or heat energy. For example, fuel 12 is contemplated to include all manner of petroleum based compositions (e.g., gasoline, diesel, heating oil, and jet fuel), agriculturally based compositions (e.g., biodiesel, ethanol, olive, soybean, cotton, rapeseed, safflower, corn and other and vegetable oils), and non-petroleum, non-agriculturally based compositions (e.g. abiogenic or deep-earth gasses). On the other hand, it is expressly contemplated that input composition 12 is broad enough to include crude oil, transformer oils, greases and other lubricants, oil contaminated with fresh, salt or brackish water, byproducts of oil production from tar sands, and “reclaimed fuels”, which is defined herein as contaminated or degraded fuel that has been returned to specification (e.g. reclaimed fuel from oil sludge at the bottom of a oil tanker tanks, old jet fuel, gasoline, diesel, etc). It is also contemplated that fuel can be “renewable”, which is defined herein as agriculturally derived fuel.

Oxidizer 14 can be any suitable oxidizing composition, including for example, hydrogen peroxide, ozone, and chromic acid. All suitable concentrations are contemplated, with concentrations and rates corresponding with experimentation. H₂O₂ is preferably employed at 10%, but at least less than 50% for safety reasons. Ozone, of course, would likely be bubbled through the mixing chamber. One advantage to H₂O₂ and ozone is that they could be generated on-site, as needed.

Additional or alternatively, oxidation can be provided electrically, through electrodes 22A,B, which are connected to a power source (not shown) through wires (not shown). Electrically produced oxidation is thought to be advantageous because it eliminates costs of transporting chemically active materials (especially strong oxidizers) to the operation site.

Catalysts 16, 18 are intended to increase or decrease the rate at which the input composition 12 is oxidized, or that preferentially oxidizes a contaminant in the input composition relative to the input composition itself. Preferred catalysts include copper, iron, magnesium, manganese, nickel, zinc, chromium or other metal ions. The cation is considered to be the catalyst, so that the anion is usually of minimal importance other than to provide a stable ionically bound substance. Exemplary catalysts include CuCl₂ and Fe₂Cl₃. FIG. 1 shows two catalysts being used, catalysts 16,18, however, those skilled in the art will appreciate that one or other numbers of catalysts can be utilized, in whatever concentrations are effective, available, and otherwise desirable.

Mixer 20 can utilize any suitable mixing technology. One of the most important parameters is whether the mixer imparts sufficient energy. Another important, but not critical parameter is that the mixer is suitable for continuous input and output flow, (as opposed to batch operation). Experimental versions have demonstrated good efficacy with sonic mixers, and critical orifice mixers. As to capacity, preferred mixers have a nominal capacity volume V, of at least 100 liters, more preferably. Full production plants are contemplated that handle 30,000,000 gallons a year (approx 113,562,353 liters/yr). For example, a 108 gallon (409 liters) mixer (V=108 gallons) at throughput of 35V≈3787 gallons/hour (14335 liters/hr) would produce about 30,000,000 gallons per year (assuming 24 hr/day operation, for 330 days a year). Such plants would, however, very likely utilize multiple lines and/or multiple mixers. Thus, the same result could obtain using two 54 gallon (204 liters) mixers.

Other nominal mixer volumes and throughput values are also contemplated. For example, mixers are contemplated having nominal capacity volumes of approximately 25 liter, 50 liter, 75 liter, 100 liter, 150 liter, and so forth. In addition, depending on the pump capacities and mixer energy density, throughputs are contemplated up to 5V, 10V, 15V, 20V, 25V, 35V, 50V, or even 100V or more.

Residence time in the mixer 20 is whatever time is needed to achieved desired mixing. In experimental devices with a small critical orifice mixer (V=1.5 liters), a suitable mixing time was about 2 minutes. Other suitable residence times in mixers are contemplated to be between about 1 minute and about 5 minutes. These and all other ranges set forth herein are inclusive of the endpoints, unless the context indicates otherwise.

Downstream of the mixer 20 is the separator 30. Separator 30 is preferably a continuous tube having a lumen with nominal volume of between 1 and 20 times that of the mixer 20. In relatively smaller mixers, such as 1 liter experimental mixers, the capacity of the separator 30 is advantageously greater, perhaps in the range of 12-18 times that of the mixer 20. Residence time in the separator 30 is whatever time is needed for the oxidizer(s) to substantially exhaust their oxidative capacity. This can be estimated by observing when the mixture seems to have stopped bubbling. In experimental devices a suitable residence time in the separator 30 is contemplated to be between about 2 minutes and about 30 minutes. Indeed, the separator 30 could be eliminated altogether (not shown), with the hydrophilic/hydrophobic compartment separations accomplished in the centrifuge, or elsewhere. In yet another alternative embodiment (not shown) a single mixer could feed multiple separators.

Filter 40 is preferably an in-line filter, with a capacity to drain off the bottoms fluid, which is the hydrophilic compartment separated out in the separator 30. Filter 40 preferably includes a disposable or regenerable physical filter, including for example, a paper or resin. Alternatively or additionally the filter 40 can include activated carbon, HRCM™ or other absorbent material. Residence time in the filter 40 is advantageously quite small, on the order of a minute or less. System 10 preferably includes dual filters 40 with an appropriate valve such that one filter can be shut down for repair, replacement, or regeneration without interrupting operation of the system.

Centrifuge 50 is intended to remove small quantities of remaining water. Any continuously operating centrifuge of sufficient capacity is acceptable. The speed and configuration should be such that water can be removed down to at most 5 ppm, more preferably at most 1 ppm, and most preferably at most 0.5 ppm. Removal of remaining water is important in many applications because the remaining water contains soluble contaminants.

Computer controller 60 has sensor feeds 62 from various sensors 62A-62E in the system 10. For example, one sensor 62A could measure whether oxidation is substantially completed, another sensor 62B could measure whether sufficient separation has occurred, another sensor 62D could detect whether the filter needs changing, and yet another sensor 62E could detect remaining impurities. Executing appropriate software (not shown), and using control wires (not shown), the controller 60 can advantageously operate pumps 19. Pumps 19 are any suitable pumps, preferably operating under the control (directly or indirectly) of the controller 60.

Thus, specific embodiments and applications of fuel purifying systems have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of marketing a fuel, comprising
providing a dirty fuel;
using a process to clean the dirty fuel to produce a cleaned fuel;
receiving written information from a competitor that the process is thought to be sufficient to reduce microbial contamination in biodiesel such that the cleaned biodiesel has no more than 100 CFUs/ml; and
qualifying the cleaned fuel under an exhaust emissions standard.

2. The method of claim 1, wherein the cleaned fuel comprises at least one of diesel, gasoline, and ethanol.

3. The method of claim 1, wherein the cleaned fuel comprises a biodiesel blend.

4. The method of claim 1, wherein the cleaned fuel contains no more than 1000 CFUs/ml of microbial contamination.

5. The method of claim 1, wherein the cleaned fuel contains no more than 100 CFUs/ml of microbial contamination.

6. The method of claim 1, wherein the cleaned fuel contains no more than 10 CFUs/ml of microbial contamination.

7. The method of claim 1, wherein the step of receiving information comprises receiving information that the process is thought to be sufficient to reduce microbial contamination in biodiesel such that the cleaned biodiesel has no more than 10 CFUs/ml.

8. The method of claim 1, further comprising testing whether the received information is correct.

9. The method of claim 1 wherein the exhaust emissions standard is at least one of: (a) a current state; and (b) a current federal standard.

10. The method of claim 1 wherein the exhaust emissions standard is a current standard, but is neither a state standard nor a federal standard.

11. The method of claim 1 wherein the process comprises washing the dirty fuel with an aqueous solution.

12. The method of claim 11 wherein the aqueous solution comprises a chemical oxidizer.

13. The method of claim 12 wherein the chemical oxidizer is selected from the group consisting of hydrogen peroxide, ozone, and chromic acid.

14. The method of claim 1 wherein the process comprises partially oxidizing the dirty fuel using electrical energy.

15. The method of claim 1 wherein the process comprises modulating oxidation of the dirty fuel using a catalyst.

16. The method of claim 15 wherein the catalyst comprises a metal ion.

17. The method of claim 16 wherein the metal ion is selected from the group consisting of copper, iron, magnesium, manganese, nickel, zinc, and chromium.

18. The method of claim 1, wherein the process comprises washing the dirty fuel with an aqueous solution including a chemical oxidizer and a catalyst, and the cleaned fuel contains no more than 100 CFUs/ml of microbial contamination.

19. The method of claim 18 wherein the process comprises sensing for an undesirable amount of a byproduct in a downstream portion of the cleaned fuel, and automatically modifying a parameter of the process to reduce the byproduct in an upstream portion of the cleaned fuel.

20. The method of claim 19 wherein the byproduct is selected from the group consisting of a triol and a glyceride.

21. A method of cleaning a fuel, comprising:
a) preparing a process fluid by combining the fuel with water and an oxidizer;
b) mixing the combination in a mixer having a volume V;
c) separating the mixed combination into hydrophobic and hydrophilic phases;
d) filtering the hydrophobic phase through a filter;
e) removing water from the filtered hydrophobic phase to produce the cleaned fuel; and
accomplishing steps a-d in a manner that produces an output of the cleaned fuel at an average rate of at least 10V/hour.

22. The method of claim 21, wherein at least one of the steps of mixing and filtering are accomplished in a substantially continuous manner.

23. The method of claim 21, wherein the steps a-e are all accomplished in a substantially continuous manner.

24. The method of claim 21, further comprising accomplishing steps a-d in a manner that produces an output of the cleaned fuel at an average rate of at least 25V/hour.

25. The method of claim 21, wherein the step of filtering comprises passing the hydrophobic phase through a stationary consumable filter material.

26. The method of claim 21, wherein the step of removing water from the filtered hydrophobic phase reduces a water content of the cleaned fuel to no more than 1 ppm.

27. The method of claim 21, wherein the step of removing water from the filtered hydrophobic phase comprising centrifuging the filtered hydrophobic phase.

28. The method of claim 21, wherein the step of filtering comprises passing the hydrophobic phase through a stationary consumable filter material, and the step of removing water from the filtered hydrophobic phase comprising centrifuging the filtered hydrophobic phase.

29. The method of claim 28 wherein the process comprises sensing for an undesirable amount of a byproduct in a downstream portion of the cleaned fuel, and automatically modifying a parameter of the process to reduce the byproduct in an upstream portion of the cleaned fuel.

30. The method of claim 29 wherein the byproduct is selected from the group consisting of a triol and a glyceride.

31. A fuel having no more than 100 CFUs/ml, that is reclaimed or renewable.

32. The fuel of claim 31 wherein the fuel comprises biodiesel.

33. The fuel of claim 31 wherein the fuel comprises ethanol.

34. The fuel of claim 31 wherein the fuel comprises no more than 5 ppm of water.

35. The fuel of claim 31 wherein the fuel has no more than 10 CFUs/ml.
